(19) United States
(12) Patent Application Publication
(10) Pub. No.: US 2025/0378943 A1
(43) Pub. Date: Dec. 11, 2025

(54) MEDICAL DEVICE REALLOCATION BASED ON USAGE DATA

(71) Applicant: Welch Allyn, Inc., Skaneateles Falls, NY (US)

(72) Inventors: Derek Strassle, Port Byron, NY (US); Kristin A. Daly, Auburn, NY (US); Anandi Mahadevan, Murrysville, PA (US)

(73) Assignee: Welch Allyn, Inc., Skaneateles Falls, NY (US)

(21) Appl. No.: 19/223,087

(22) Filed: May 30, 2025

Related U.S. Application Data
(60) Provisional application No. 63/658,617, filed on Jun. 11, 2024.

(51) Int. Cl. G16H 40/40; G16H 40/20

(52) U.S. Cl. CPC G16H 40/40; G06Q 10/06313; G16H 40/20

(57) ABSTRACT

A diagnostic monitoring system for a medical device in a healthcare facility includes a service platform that receive usage data from the medical device and determines a diagnostic status of the medical device based on the received usage data. The service platform triggers a notification indicating whether the medical device should be reallocated to a different location in the healthcare facility based on the diagnostic status.

### CROSS-REFERENCE TO RELATED APPLICATIONS

This application claims priority under 35 U.S.C. § 119 (e) to U.S. Provisional Application No. 63/658,617, filed Jun. 11, 2024, which is expressly incorporated by reference herein.

### BACKGROUND

present disclosure relates to a monitoring system for a medical device, and particularly, to medical device reallocation based on usage data.

Medical devices, for example, patient support systems such as hospital beds and patient monitoring systems, include many components and associated equipment which are subject to a limited predetermined number of cycles, wear and tear, and which require preventative or responsive service or replacement. In some cases such service or replacement is determined or even predicted by monitoring the data available from a sensor system associated with the medical device. For example, as a fault condition occurs or as the predetermined limited number of cycles of an actuator or other component is being approached, preventive service or replacement of the actuator or other component is indicated. In some situations, certain medical devices within a healthcare facility are used more often than other devices. Generally, the devices in the healthcare facility are not regularly reallocated to account for discrepancies in use. As a result, some areas of the healthcare facility have devices that reach the need for replacement or service earlier than other devices.

### SUMMARY

The present disclosure includes one or more of the features recited in the appended claims and/or the following features which, alone or in any combination, may comprise patentable subject matter.

According to a first aspect of the disclosed embodiments, a diagnostic monitoring system for a medical device in a healthcare facility includes a service platform configured to receive usage data relating to at least one parameter of the medical device and determine a diagnostic status of the medical device based on the received usage data. The service platform triggers a notification indicating whether the medical device should be reallocated to a different location in the healthcare facility based on the diagnostic status. A display displays the notification that the medical device should be reallocated to different location in the healthcare facility.

In some embodiments of the first aspect, the usage data may include a cycle count of the medical device. The usage data may include a total run time of the medical device. The diagnostic status may indicate that the medical device has reached half of an expected life based on at least one of a cycle count and a total run time of the medical device. The diagnostic status may indicate that the device is approaching an end of an expected life based on at least one of a cycle count and a total run time of the medical device. The diagnostic status may indicate that the device is being used at a higher rate than an average rate of usage based on at least one of a cycle count and a total run time of the medical device. The notification may indicate that the medical device should be reallocated to a location in the healthcare facility where the medical device will experience a lower rate of usage based on at least one of a cycle count and a total run time of the medical device. The medical device may be a first medical device. The notification may indicate a second medical device to swap with the first medical device. The notification may indicate a location of the second medical device. The service platform may be located remotely from the medical device.

According to a second aspect of the disclosed embodiments, a method for reallocating a medical device in a healthcare facility includes generating usage data relating to at least one parameter of the medical device. The method also includes transmitting the usage data to a service platform to determine a diagnostic status of the medical device based on the usage data. The method also includes triggering a notification indicating whether the medical device should be reallocated to a different location in the healthcare facility based on the diagnostic status. The method also includes displaying the notification that the medical device should be reallocated to different location in the healthcare facility.

In some embodiments of the second aspect, the usage data may include a cycle count of the medical device. The usage data may include a total run time of the medical device. The method may include indicating that the medical device has reached half of an expected life based on at least one of a cycle count and a total run time of the medical device. The method may include indicating that the device is approaching an end of an expected life based on at least one of a cycle count and a total run time of the medical device. The method may include indicating that the device is being used at a higher rate than an average rate of usage based on at least one of a cycle count and a total run time of the medical device. The method may include indicating that the medical device should be reallocated to a location in the healthcare facility where the medical device will experience a lower rate of usage based on at least one of a cycle count and a total run time of the medical device. The medical device may be a first medical device. The method may include indicating a second medical device to swap with the first medical device. The method may include indicating a location of the second medical device. The service platform may be located remotely from the medical device.

Additional features, which alone or in combination with any other feature(s), such as those listed above and those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a diagrammatic view of a diagnostic monitoring system for a medical device in a healthcare facility, in accordance with an embodiment;

FIG. 2 is a view of an exemplary screenshot displayed on a display of the system shown in FIG. 1; and

FIG. 3 is a flowchart of a method for reallocating a medical device in the healthcare facility.

### DETAILED DESCRIPTION

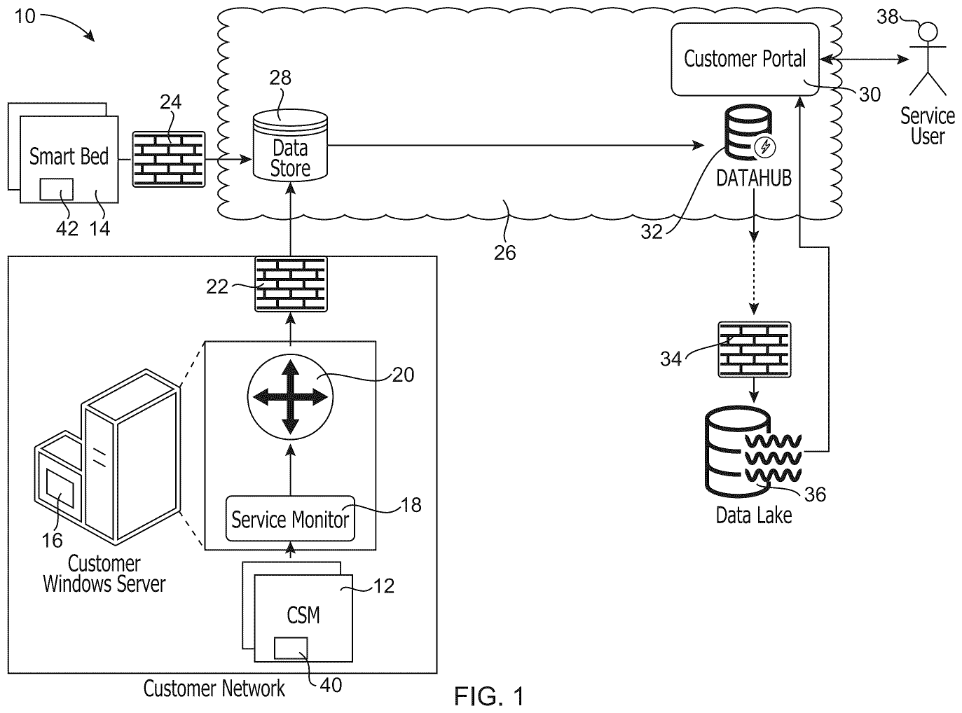

Referring to FIG. 1, a diagnostic monitoring system 10 for medical devices 12, 14 in a healthcare facility includes a service platform or network 26 that is configured for remote diagnostics of the medical devices 12, 14. In the exemplary embodiment, the medical device 14 is a patient support apparatus illustrated as a bed and the medical device 12 is a monitor used in a healthcare facility. Patient monitors/devices 14 include, for example, heart rate (HR) monitors, respiration rate (RR) monitors, pulse oximeters for measuring blood oxygenation percentages (aka SpO2), thermometers to measure patient temperature, blood pressure devices, electrocardiograms (EKG) devices, electroencephalograms (EEG) devices, IV pumps, hemodialysis machines, and patient lifts, just to name a few. In some embodiments, patient monitors 14 include a pair of CONNEX® Vital Signs Monitors (CVSM's) which are available from Welch Allyn, Inc. of Skaneateles Falls, New York and a pair of CONNEX® Spot Monitors (CSM's) which are also available from Welch Allyn, Inc.

It should be readily appreciated that the facility may include multiple Local Area Networks (“LANs”) and/or Wide Area Networks (“WANs”) that are operably coupled to one another via routers, switches, hubs, gateways, proxies, and/or firewalls 22, 24, and 34. The service platform 26 is configured to enable remote diagnostics of the medical devices 12, 14. For example, the service platform 26 recognizes the medical devices 12, 14 and stores unique data, e.g. a serial number, model number, etc in a database 28. A sensor 40 is provided in the medical device 12 to collect usage data related to the medical device 12. Likewise, a sensor 42 is provided in the medical device 14 to collect usage data related to the medical device 14. In some embodiments, the sensors 40, 42 are monitors, internal computer processing units, or other devices capable of tracking usage data of the devices 12, 14. The usage data includes a cycle count and a total run time of the respective medical device 12, 14. The usage data is also stored in the database 28. In the example of the medical device 12, the usage data is transmitted to the database 28 through a customer (healthcare facility) server 16 that includes middleware gateway components 18, 20 that reside on the customer server 16 for the devices 12 to communicate with the service platform 26.

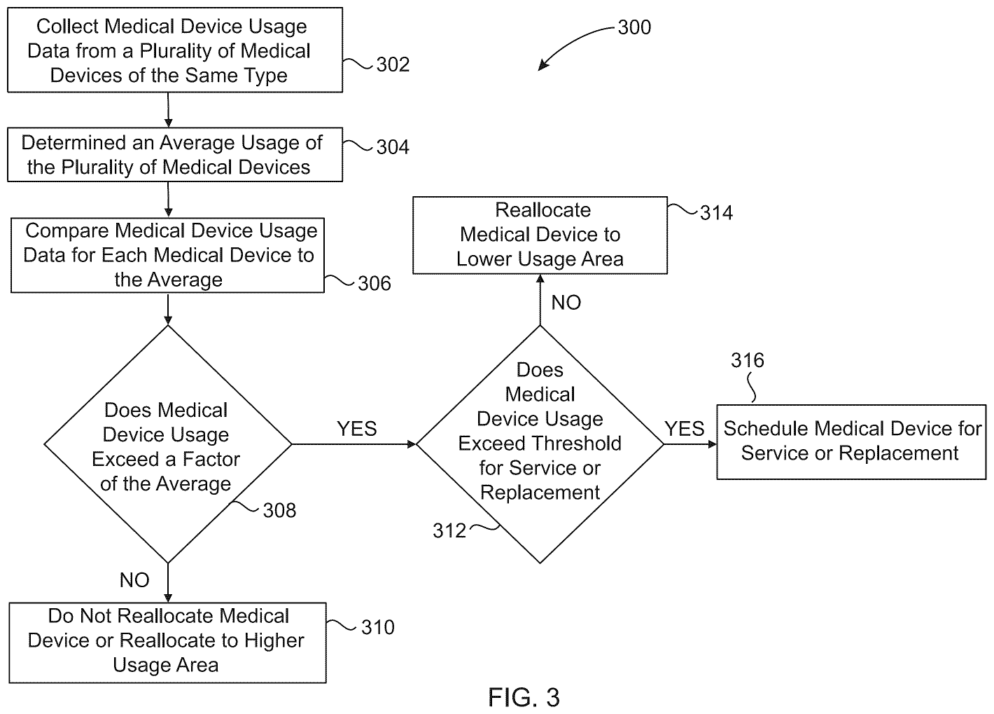

The service platform 26 is located remotely from the healthcare facility for remote service. A customer portal application 30 resides in the service platform 26. The medical devices 12, 14 connect to the service platform 26 and periodically send the usage data to the database 28. The usage data is then sent to an application 32 application on the service platform 26 that extracts the data within the database 28 and offloads it to the data lake 36. In the data lake 36 the usage data is used to source dashboards that are hosted as embedded frames within the customer portal application 30. Service users 38 interact with the dashboard content from the customer portal 30 to determine whether the medical devices 12, 14 should be swapped with other similar devices based on the usage data. A method for determining whether the medical devices 12, 14 should be swapped with other devices in set forth in FIG. 3. In some embodiments, the usage date is also used to determine whether the medical devices 12, 14 should be replaced or serviced.

The above described components associated with service platform 26 for which diagnostic information is provided to the monitoring device 38 are illustrative and not intended to be an all-inclusive list. Other components for which diagnostic information can be provided include, for example, drives, including motors, pumps, and actuators; user controls, including indicators and switches for the bed, lighting, entertainment, and nurse call; caregiver controls, including indicators and switches for bed positioning, drives, brakes, air systems, and other therapeutic systems; air systems, including compressors, blowers, bladders, and valves; movable members, including surfaces such as for the head, leg, knee, foot, elevation, and tilt, side rails, wheel systems, surface drive systems, braking systems, and linkages; power supplies, including battery voltage, charge state, charge current, discharge, and capacity; electronics, including control logic and communication circuits; load cells; and sensors for all of the above. The diagnostic information that is obtained and transmitted to the service platform 26 can include or be related to vibration, displacement, rate, component temperature, ambient temperature, component humidity, ambient humidity, thermal loading, pressure, noise, mechanical load, current, voltage, electrical power, signal signature, calibration values, transit time, fault rate, logic or communication error, and accumulated usage, for example, the number of cycles or elapsed time compared to a predefined threshold.

The service platform 26 is configured to collect, monitor, and analyze the diagnostic information to predict and determine needed component reallocation, replacement, or service. The service platform 26 can be configured to compare diagnostic information to predefined thresholds and can additionally or alternatively use more complex analysis. For example, in order to identify and report preventative or responsive service requirements, the service platform 26 can use real-time and historical data, probabilistic methods, performance trends of an individual device and/or relative to other devices, and thresholds for diagnostic information.

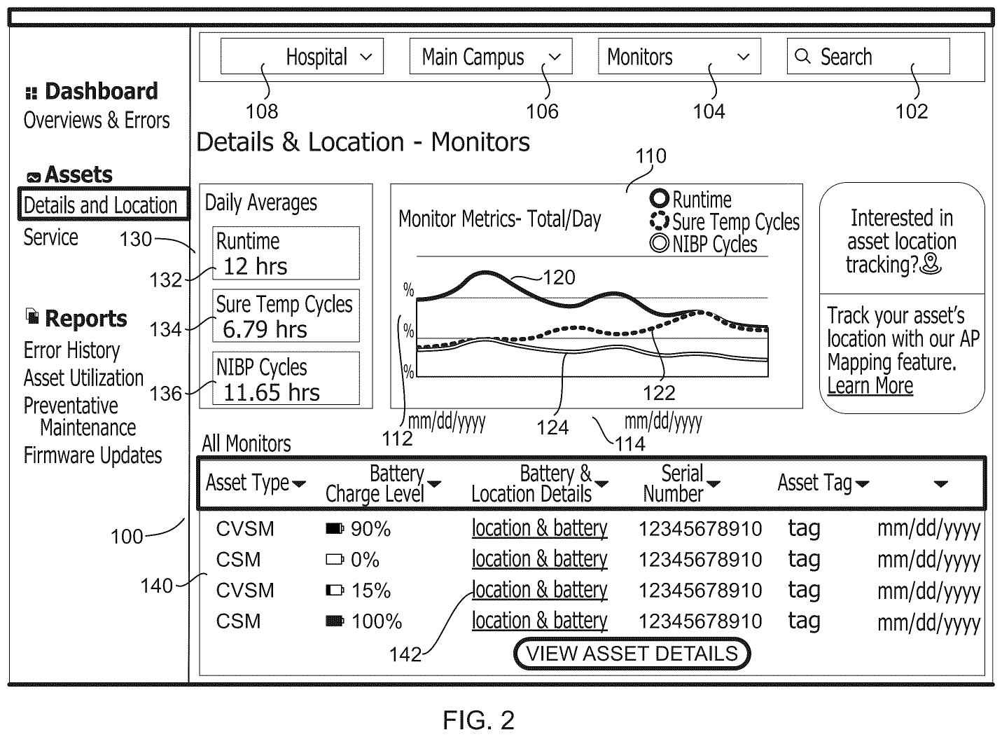

FIG. 2 illustrates a screenshot 100 of the dashboard of the customer portal 30 that displays the usage data of a medical device 12. Although FIG. 2 is described with respect to medical devices 12 including monitors, it will be appreciated that a similar display is provided for patient support apparatuses, i.e. medical devices 14. A top ribbon 102 of the display 100 includes data related to the healthcare facility. For example, the illustrative embodiment, displays monitors 104 at the main campus 106 of a hospital 108.

A graph 110 illustrates run time and cycles 112 of the medical device 12 over time 114 (shown in days). A first line 120 illustrates a total runtime of the medical device 12. A second line 122 illustrates a number of cycles that a thermometer of the medical device 12 has been used. A third line 124 illustrates a number of cycles that the non-invasive blood pressure monitor of the medical device 12 has been used. A table 130 illustrates average runtime 132 of the medical device 12 per day, an average a number of cycles 134 that the thermometer of the medical device 12 has been used per day, and an average number of cycles 136 that the non-invasive blood pressure monitor of the medical device 12 has been used per day. It should be noted that the numbers illustrated in the graph 110 and the table 130 are exemplary only for the purposes of illustrating the type of usage data displayed by the dashboard of the customer portal 30.

In the event that the medical device 12 requires reallocation, the display 100 provides a list 140 of other similar medical devices that can be swapped with the medical device 12. The list 140 includes a link 142 for each respective device. By clicking on the link 142 a location of the other device in the facility and a status of the other device's remaining battery power is displayed on the dashboard of the customer portal 30.

Referring now to FIG. 3, a flowchart 300 illustrates a method for reallocating the medical devices 12, 14 in the healthcare facility. At block 302, the sensors 40, 42 collect usage data related to a plurality of medical devices 12, 14. In some embodiments, the usage data is related to a cycle count or a total run time of the medical devices 12, 14. It will be appreciated that, in some embodiments, the usage data includes a total time that the medical devices 12, 14 has been in service. At block 304, the service platform 26 determines an average usage of the plurality of medical devices 12, 14. At block, 306, the service platform 26 compares the medical device usage data for each medical device 12, 14 to the average.

At block 308, the service platform 26 determines whether the usage of the medical devices 12, 14 has reached or exceeded a predetermined limit. For example, in an exemplary embodiment, the predetermined limit is half of an expected life of the medical devices 12, 14 based on the usage data of the medical devices 12, 14. In another embodiment, the service platform 26 determines whether the medical devices 12, 14 are being used at a higher rate than an average rate of usage for similar medical devices. In one embodiment, the service platform 26 determines if the usage of the medical devices 12, 14 has exceed a factor of the average usage, for example 1.5 times the average usage. If the medical devices 12, 14 have not reached the predetermined limit, at block 310, the medical devices 12, 14 are not reallocated within the healthcare facility. In some embodiments, if the medical devices 12, 14 have not reached the predetermined limit, the medical devices 12, 14 are flagged for potential reallocation with another overused device. That is, the medical devices 12, 14 are flagged for potential swapping with an overused device in the future. In some embodiments, the medical devices 12, 14 are reallocated to a higher usage area if the usage of the medical devices 12, 14 is below a predetermined threshold, for example, 0.5 times the average usage. In some embodiments, the service platform 26 tracks how often a medical device 12, 14 has been reallocated to avoid frequent reallocations of the same medical device 12, 14.

If the medical devices 12, 14 have reached the predetermined limit, at block 312, the service platform 26 determines whether the medical devices 12, 14 usage exceeds a threshold for service or replacement. For example, in some embodiments, the medical devices 12, 14 exceed the threshold when usage of the medical devices 12, 14 exceeds a predetermined percentage of the expected life of the medical devices 12, 14. In some embodiments, the predetermined percentage is between 80% and 95% of the expected life span. If the medical devices 12, 14 are not determined to exceed the threshold, at block 314, the medical devices 12, 14 are reallocated to an area of the healthcare facility where the medical devices 12, 14 will experience lower usage. In some embodiments, the service platform 26 sends a notification that is displayed on the dashboard of the customer portal 30 that the medical devices 12, 14 should be reallocated. In some embodiments, the notification indicates a location in the healthcare facility of a second medical device with which the medical devices 12, 14 should be swapped. In some embodiments, the second medical device is another similar device with low usage that has been previously flagged for reallocation with a high usage device. If the medical devices 12, 14 are determined to exceed the threshold, at block 316, the medical devices 12, 14 are scheduled for repair or replacement. In some embodiments, the service platform 26 sends a notification that is displayed on the dashboard of the customer portal 30 that the medical devices 12, 14 should be scheduled for repair or replacement. In some embodiments, the service platform 26 sends a notification that is displayed on the dashboard of the customer portal 30 that a new medical device should be ordered. In some embodiments, a sales representative is notified that the medical devices 12, 14 should be scheduled for repair or replacement.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used, the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles have been presented, they need not be utilized in combination, and many combinations of aspects and principles are possible in light of the various embodiments provided above.

1. A diagnostic monitoring system for a medical device in a healthcare facility, the diagnostic monitoring system comprising:
a service platform configured to receive usage data relating to at least one parameter of the medical device and determine a diagnostic status of the medical device based on the received usage data, wherein the service platform triggers a notification indicating whether the medical device should be reallocated to a different location in the healthcare facility based on the diagnostic status, and
a display for displaying the notification that the medical device should be reallocated to the different location in the healthcare facility.

2. The system of claim 1, wherein the usage data includes a cycle count of the medical device.

3. The system of claim 1, wherein the usage data includes a total run time of the medical device.

4. The system of claim 1, wherein the diagnostic status indicates that the medical device has reached half of an expected life based on at least one of a cycle count and a total run time of the medical device.

5. The system of claim 1, wherein the diagnostic status indicates that the device is approaching an end of an expected life based on at least one of a cycle count and a total run time of the medical device.

6. The system of claim 1, wherein the diagnostic status indicates that the device is being used at a higher rate than an average rate of usage based on at least one of a cycle count and a total run time of the medical device.

7. The system of claim 1, wherein the notification indicates that the medical device should be reallocated to a location in the healthcare facility where the medical device will experience a lower rate of usage based on at least one of a cycle count and a total run time of the medical device.

8. The system of claim 1, wherein:
the medical device is a first medical device, and
the notification indicates a second medical device to swap with the first medical device.

9. The system of claim 8, wherein the notification indicates a location of the second medical device.

10. The system of claim 1, wherein the service platform is located remotely from the medical device.

11. A method for reallocating a medical device in a healthcare facility, the method comprising:
generating usage data relating to at least one parameter of the medical device, transmitting the usage data to a service platform to determine a diagnostic status of the medical device based on the usage data,
triggering a notification indicating whether the medical device should be reallocated to a different location in the healthcare facility based on the diagnostic status, and
displaying the notification that the medical device should be reallocated to the different location in the healthcare facility.

12. The method of claim 11, wherein the usage data includes a cycle count of the medical device.

13. The method of claim 11, wherein the usage data includes a total run time of the medical device.

14. The method of claim 11, further comprising indicating that the medical device has reached half of an expected life based on at least one of a cycle count and a total run time of the medical device.

15. The method of claim 11, further comprising indicating that the device is approaching an end of an expected life based on at least one of a cycle count and a total run time of the medical device.

16. The method of claim 11, further comprising indicating that the device is being used at a higher rate than an average rate of usage based on at least one of a cycle count and a total run time of the medical device.

17. The method of claim 11, further comprising indicating that the medical device should be reallocated to a location in the healthcare facility where the medical device will experience a lower rate of usage based on at least one of a cycle count and a total run time of the medical device.

18. The method of claim 11, wherein the medical device is a first medical device, and the method further includes indicating a second medical device to swap with the first medical device.

19. The method of claim 18, further comprising indicating a location of the second medical device.

20. The method of claim 11, wherein the service platform is located remotely from the medical device.
